(19) United States
(12) Patent Application Publication
(10) Pub. No.: US 2006/0293292 A1
(43) Pub. Date: Dec. 28, 2006

(54) METHODS OF USING ACYL HYDRAZONES AS sEH INHIBITORS

(75) Inventors: Mario G. CARDOZO, San Francisco, CA (US); Richard Harold INGRAHAM, New Fairfield, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim International GmbH, Ingelheim (DE)

(21) Appl. No.: 11/380,999

(22) Filed: May 1, 2006

Related U.S. Application Data
(60) Provisional application No. 60/678,871, filed on May 6, 2005.

(51) Int. Cl. A61K 31/57; A61K 31/537; A61K 31/495; A61K 31/4706; A61K 31/445; A61K 31/44; A61K 31/426; A61K 31/415; A61K 31/4172; A61K 31/41; A61K 31/40; A61K 31/405; A61K 31/175

(52) U.S. Cl. 514/169; 514/237.5; 514/255.01; 514/313; 514/317; 514/352; 514/408; 514/424; 514/400; 514/419; 514/370; 514/471; 514/381; 514/452; 514/467; 514/590; 514/614

(57) ABSTRACT

Disclosed are hydrazine compounds useful as soluble epoxide hydrolase (sEH) inhibitors for treating cardiovascular diseases.

### APPLICATION DATA

This application claims benefit to U.S. provisional application Ser. No. 60/678,871 filed May 6, 2005.

### FIELD OF THE INVENTION

This invention is directed to methods of using soluble epoxide hydrolase (sEH) inhibitors for diseases related to cardiovascular disease.

### BACKGROUND OF THE INVENTION

Epoxide hydrolases are a group of enzymes ubiquitous in nature, detected in species ranging from plants to mammals. These enzymes are functionally related in that they all catalyze the addition of water to an epoxide, resulting in a diol. Epoxide hydrolases are important metabolizing enzymes in living systems. Epoxides are reactive species and once formed are capable of undergoing nucleophilic addition. Epoxides are frequently found as intermediates in the metabolic pathway of xenobiotics. Thus in the process of metabolism of xenobiotics, reactive species are formed which are capable of undergoing addition to biological nucleophiles. Epoxide hydrolases are therefore important enzymes for the detoxification of epoxides by conversion to their corresponding, non-reactive diols.

In mammals, several types of epoxide hydrolases have been characterized including soluble epoxide hydrolase (sEH), also referred to as cytosolic epoxide hydrolase, cholesterol epoxide hydrolase, LTA₄ hydrolase, hepoxilin hydrolase, and microsomal epoxide hydrolase (Fretland and Omiecinski, Chemico-Biological Interactions, 129: 41-59 (2000)). Epoxide hydrolases have been found in all tissues examined in vertebrates including heart, kidney and liver (Vogel, et al., Eur J. Biochemistry, 126: 425-431 (1982); Schladt et al., Biochem. Pharmacol., 35: 3309-3316 (1986)). Epoxide hydrolases have also been detected in human blood components including lymphocytes (e.g. T-lymphocytes), monocytes, erythrocytes, platelets and plasma. In the blood, most of the sEH detected was present in lymphocytes (Seidegard et al., Cancer Research, 44: 3654-3660 (1984)).

The epoxide hydrolases differ in their specificity towards epoxide substrates. For example, sEH is selective for aliphatic epoxides such as epoxide fatty acids while microsomal epoxide hydrolase (mEH) is more selective for cyclic and arene oxides. The primary known physiological substrates of sEH are four regioisomeric cis epoxides of arachidonic acid known as epoxyeicosatrienoic acids or EETs. These are 5,6-, 8,9-, 11,12-, and 14,15-epoxyeicosatrienoic acid. Also known to be substrates are epoxides of linoleic acid known as leukotoxin or isoleukotoxin. Both the EETs and the leukotoxins are generated by members of the cytochrome P450 monooxygenase family (Capdevila, et al., J. Lipid Res., 41: 163-181 (2000)).

The various EETs appear to function as chemical mediators that may act in both autocrine and paracrine roles. EETs appear to be able to function as endothelial derived hyperpolarizing factor (EDHF) in various vascular beds due to their ability to cause hyperpolarization of the membranes of vascular smooth muscle cells with resultant vasodilation (Weintraub, et al., Circ. Res., 81: 258-267 (1997)). EDHF is synthesized from arachidonic acid by various cytochrome P450 enzymes in endothelial cells proximal to vascular smooth muscle (Quilley, et al., Brit. Pharm., 54: 1059 (1997)); Quilley and McGiff, TIPS, 21: 121-124 (2000)); Fleming and Busse, Nephrol. Dial. Transplant, 13: 2721-2723 (1998)). In the vascular smooth muscle cells EETs provoke signaling pathways which lead to activation of BK_{Ca2} channels (big Ca²⁺ activated potassium channels) and inhibition of L-type Ca²⁺ channels. This results in hyperpolarization of membrane potential, inhibition of Ca²⁺ influx and relaxation (Li et al., Circ. Res., 85: 349-356 (1999)). Endothelium dependent vasodilation has been shown to be impaired in different forms of experimental hypertension as well as in human hypertension (Lind, et al., Blood Pressure, 9: 4-15 (2000)). Impaired endothelium dependent vasorelaxation is also a characteristic feature of the syndrome known as endothelial dysfunction (Goligorsky, et. al., Hypertension, 37[part 2]: 744-748 ( 2001). Endothelial dysfunction plays a significant role in a large number of pathological conditions including type 1 and type 2 diabetes, insulin resistance syndrome, hypertension, atherosclerosis, coronary artery disease, angina, ischemia, ischemic stroke, Raynaud's disease and renal disease. Hence, it is likely that enhancement of EETs concentration would have a beneficial therapeutic effect in patients where endothelial dysfunction plays a causative role. Other effects of EETs that may influence hypertension involve effects on kidney function. Levels of various EETs and their hydrolysis products, the DHETs, increase significantly both in the kidneys of spontaneously hypertensive rats (SHR) (Yu, et al., Circ. Res. 87: 992-998 (2000)) and in women suffering from pregnancy induced hypertension (Catella, et al., Proc. Natl. Acad. Sci. U.S.A., 87: 5893-5897 (1990)). In the spontaneously hypertensive rat model, both cytochrome P450 and sEH activities were found to increase (Yu et al., Molecular Pharmacology, 2000, 57, 1011-1020). Addition of a known sEH inhibitor was shown to decrease the blood pressure to normal levels. Finally, male soluble epoxide hydrolase null mice exhibited a phenotype characterized by lower blood pressure than their wild-type counterparts (Sinal, et al., J. Biol. Chem., 275: 40504-40510 (2000)).

EETs, especially 11,12-EET, also have been shown to exhibit anti-inflammatory properties (Node, et al., Science, 285: 1276-1279 (1999); Campbell, TIPS, 21: 125-127 (2000); Zeldin and Liao, TIPS, 21: 127-128 (2000)). Node, et al. have demonstrated 11,12-EET decreases expression of cytokine induced endothelial cell adhesion molecules, especially VCAM-1. They further showed that EETs prevent leukocyte adhesion to the vascular wall and that the mechanism responsible involves inhibition of NF-κB and IκB kinase. Vascular inflammation plays a role in endothelial dysfunction (Kessler, et al., Circulation, 99: 1878-1884 (1999)). Hence, the ability of EETs to inhibit the NF-κB pathway should also help ameliorate this condition.

In addition to the physiological effect of some substrates of sEH (EETs, mentioned above), some diols, i.e. DHETs, produced by sEH may have potent biological effects. For example, sEH metabolism of epoxides produced from linoleic acid (leukotoxin and isoleukotoxin) produces leukotoxin and isoleukotoxin diols (Greene, et al., Arch. Biochem. Biophys. 376(2): 420-432 (2000)). These diols were shown to be toxic to cultured rat alveolar epithelial cells, increasing intracellular calcium levels, increasing intercellular junction permeability and promoting loss of epithelial integrity (Moghaddam et al., Nature Medicine, 3: 562-566 (1997)). Therefore these diols could contribute to the etiology of diseases such as adult respiratory distress syndrome where lung leukotoxin levels have been shown to be elevated (Ishizaki, et al., Pulm. Pharm. & Therap., 12: 145-155 (1999)). Hammock, et al. have disclosed the treatment of inflammatory diseases, in particular adult respiratory distress syndrome and other acute inflammatory conditions mediated by lipid metabolites, by the administration of inhibitors of epoxide hydrolase (WO 98/06261; U.S. Pat. No. 5,955,496).

A number of classes of sEH inhibitors have been identified. Among these are chalcone oxide derivatives (Miyamoto, et al. Arch. Biochem. Biophys., 254: 203-213 (1987)) and various trans-3-phenylglycidols (Dietze, et al., Biochem. Pharm. 42: 1163-1175 (1991); Dietze, et al., Comp. Biochem. Physiol. B, 104: 309-314 (1993)).

More recently, Hammock et al. have disclosed certain biologically stable inhibitors of sEH for the treatment of inflammatory diseases, for use in affinity separations of epoxide hydrolases and in agricultural applications (U.S. Pat. No. 6,150,415). The Hammock '415 patent also generally describes that the disclosed pharmacophores can be used to deliver a reactive functionality to the catalytic site, e.g., alkylating agents or Michael acceptors, and that these reactive functionalities can be used to deliver fluorescent or affinity labels to the enzyme active site for enzyme detection (col. 4, line 66 to col. 5, line 5). Certain urea and carbamate inhibitors of sEH have also been described in the literature (Morisseau et al., Proc. Natl. Acad. Sci., 96: 8849-8854 (1999); Argiriadi et al., J. Biol. Chem., 275 (20) 15265-15270 (2000); Nakagawa et al. Bioorg. Med. Chem., 8: 2663-2673 (2000)).

WO 99/62885 (A1) discloses 1-(4-aminophenyl)pyrazoles having anti-inflammatory activity resulting from their ability to inhibit IL-2 production in T-lymphocytes, it does not however, disclose or suggest compounds therein being effective inhibitors of sEH. WO 00/23060 discloses a method of treating immunological disorders mediated by T-lymphocytes by administration of an inhibitor of sEH. Several 1-(4-aminophenyl)pyrazoles are given as examples of inhibitors of sEH.

U.S. Pat. No. 6,150,415 to Hammock is directed to a method of treating an epoxide hydrolase, using compounds having the structure 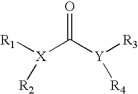
wherein X and Y is each independently nitrogen, oxygen, or sulfur, and X can further be carbon, at least one of R1-R4 is hydrogen, R2 is hydrogen when X is nitrogen but is not present when X is sulfur or oxygen, R4 is hydrogen when Y is nitrogen but is not present when Y is sulfur or oxygen, R1 and R3 is each independently H, C1-20 substituted or unsubstituted alkyl, cycloalkyl, aryl, acyl, or heterocyclic. Related to the Hammock patent is U.S. Pat. No. 6,531,506 to Kroetz et al. which claims a method of treating hypertension using of an inhibitor of epoxide hydrolase, also claimed are methods of treating hypertension using compounds similar to those described in the Hammock patent. Neither of these patents teaches or suggests methods of treating cardiovascular diseases using the particular sEH inhibitors described herein.

As outlined in the discussion above, inhibitors of sEH are useful therefore, in the treatment of cardiovascular diseases such as endothelial dysfunction either by preventing the degradation of sEH substrates that have beneficial effects or by preventing the formation of metabolites that have adverse effects.

All references cited above and throughout this application are incorporated herein by reference in their entirety.

### SUMMARY OF THE INVENTION

It is therefore an object of the invention to provide a method of treating a cardiovascular disease; said method comprising administering to a patient in need thereof a therapeutically effective amount of compounds as listed herein below.

### DETAILED DESCRIPTION OF THE INVENTION

In a broad generic aspect of the invention, there is provided a method of treating a cardiovascular disease, said method comprising administering to a patient in need thereof a therapeutically effective amount of a compounds of the formulas (I) or (II):

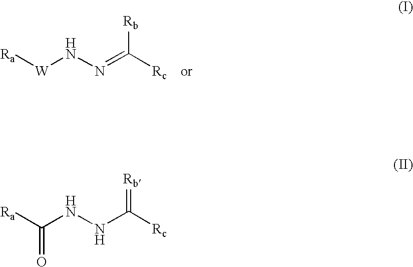

W is a bond or >C═O;

Rₐ and R_{c} are
—Ar₁, —(Ar₂)ₜ—(CH₂)_{q}—X₁—(CH₂)ₙ—X₂—(CH₂)ₘ—Y wherein Y is —CH₃ or Ar₁,
Ar₁ and Ar₂ are each independently a heterocylic or carbocyclic ring system, each X₁ and X₂ are independently a bond, >C═O, O, NH, NR or S(O)ₚ;
m, n and q are 0-5;
t is 0 or 1;
p is 0-2;
each alkyl chain formed by —(CH₂)_{q}—, —(CH₂)ₙ—, —(CH₂)ₘ— can be saturated or partially or fully unsaturated;
R_{b}′ is ═O, ═NH, ═CH₂,
R_{b} is hydrogen or C₁₋₅ alkyl,
or R_{b} and R_{c} fuse to form a 3-17 carbon carbocyclic or a 4-17 carbon heterocyclic ring system, each ring system being mono-, bicyclic-,tricyclic or tetracyclic
each of the aforementioned rings in this embodiment is optionally substituted by one or more halogen, nitro, amine, C₁₋₅ alkyl, C₁₋₅ alkoxy or hydroxyl;
or the pharmaceutically acceptable salts thereof

Preferred Ar₁ and Ar₂ include:

Phenyl, thienyl, morpholine, quinoline, piperidine, bicycle[2.2.1]heptane, adamantly, pyrrolidine, pyrrolidinone, pyridine, naphthylene, benzthiophene, furan, imidazole, benzodioxolanyl, benzodioxanyl, indole, indane, piperazine, thiazole, pyrimidine, cyclopropyl, cyclobutyl, cyclopentyl, cyclohexyl, tetrazole 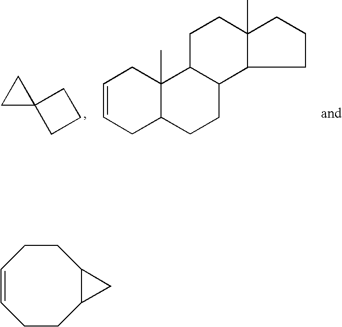
each Ar₁ and Ar₂ is optionally substituted by one or more halogen, nitro, amine, C₁₋₅ alkyl, C₁₋₅ alkoxy or hydroxyl.

In another embodiment of the invention, there is provided a method of treating a cardiovascular disease, said method comprising administering to a patient in need thereof a therapeutically effective amount of one or more compounds chosen from: 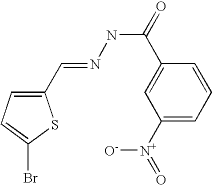 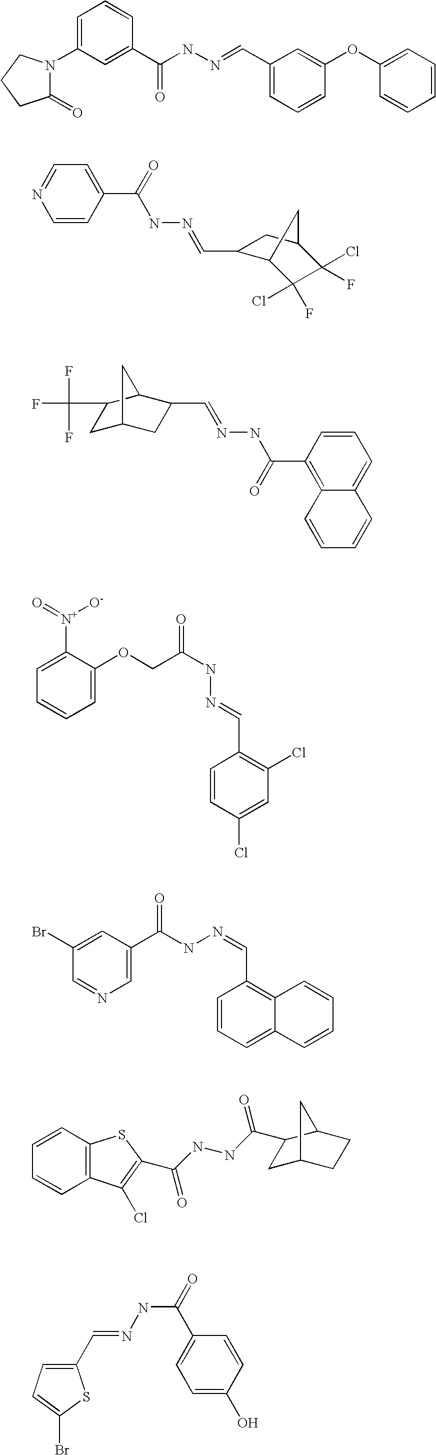 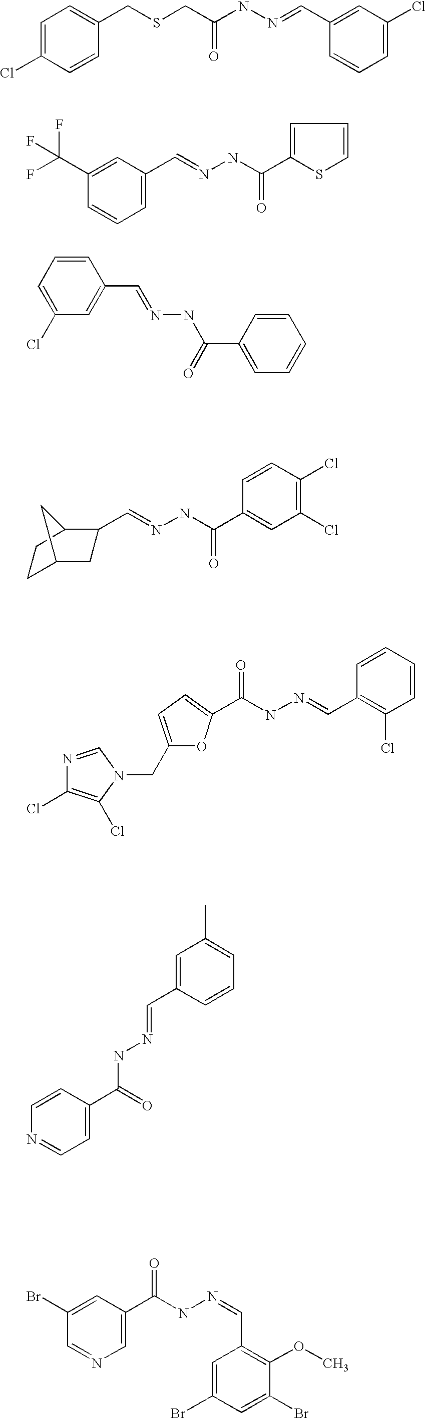 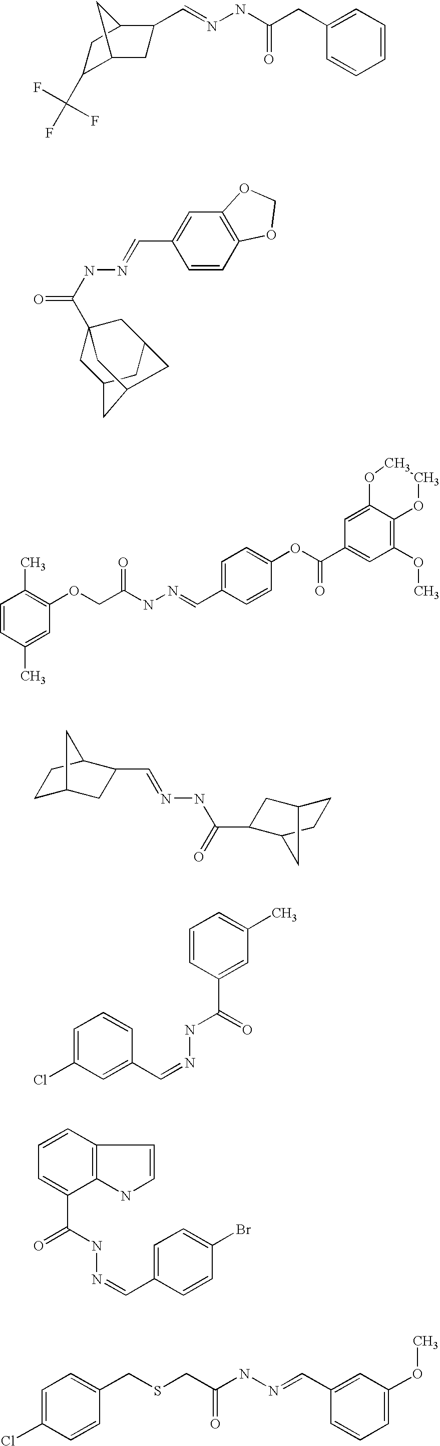 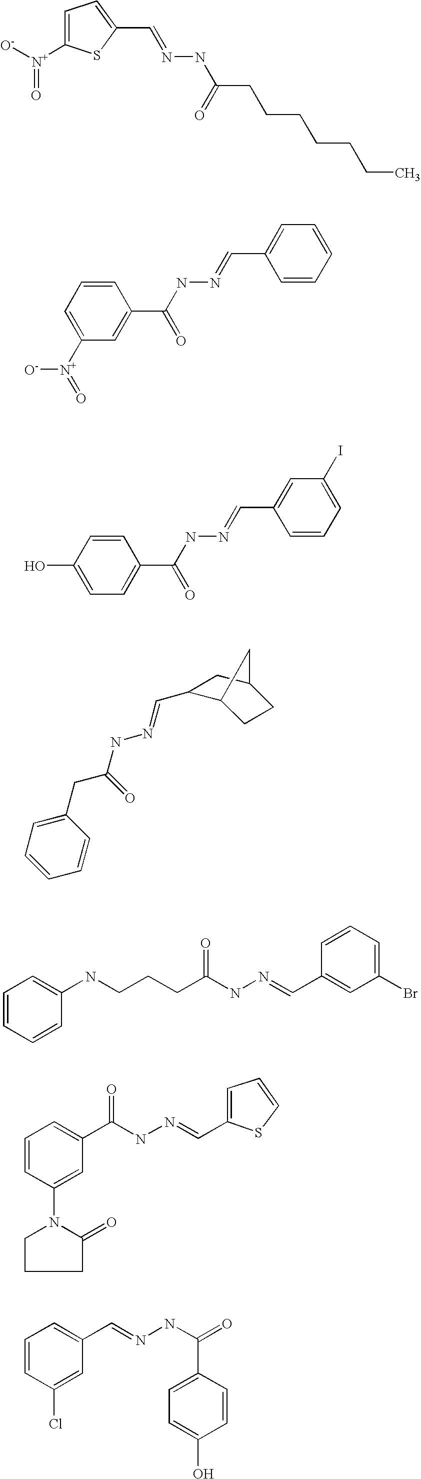 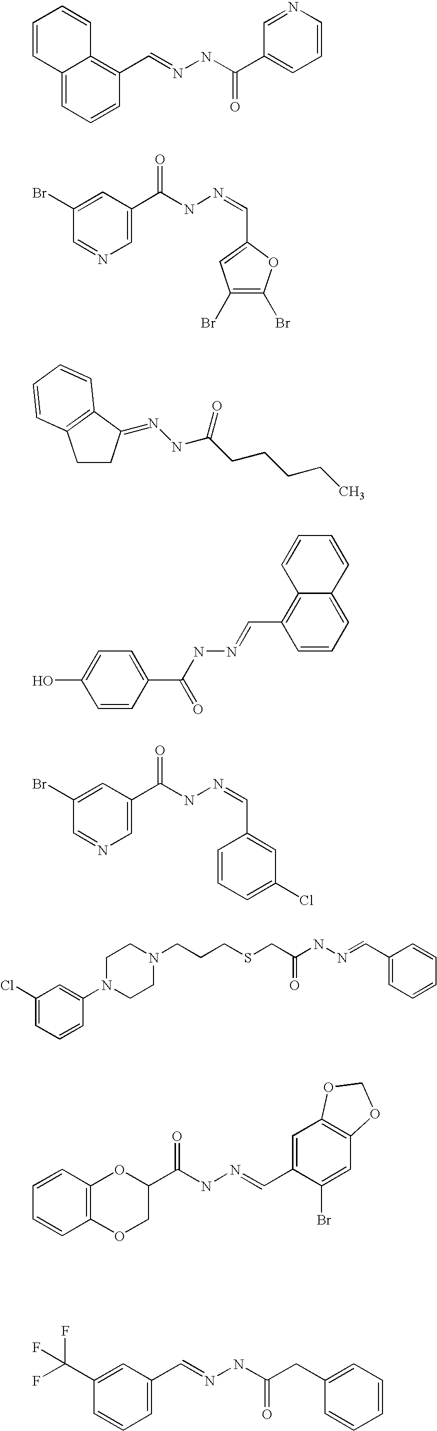 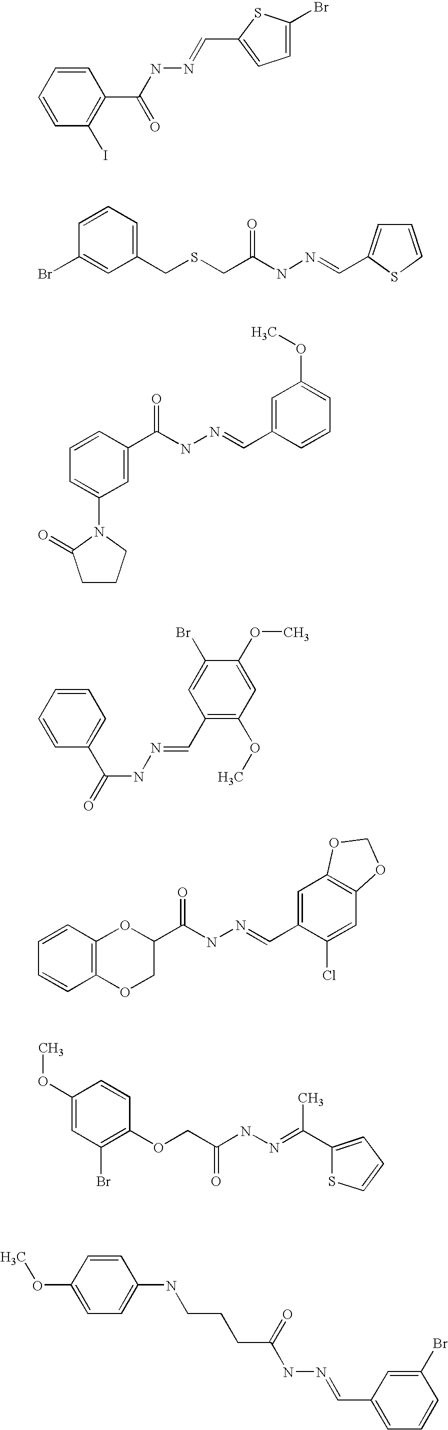 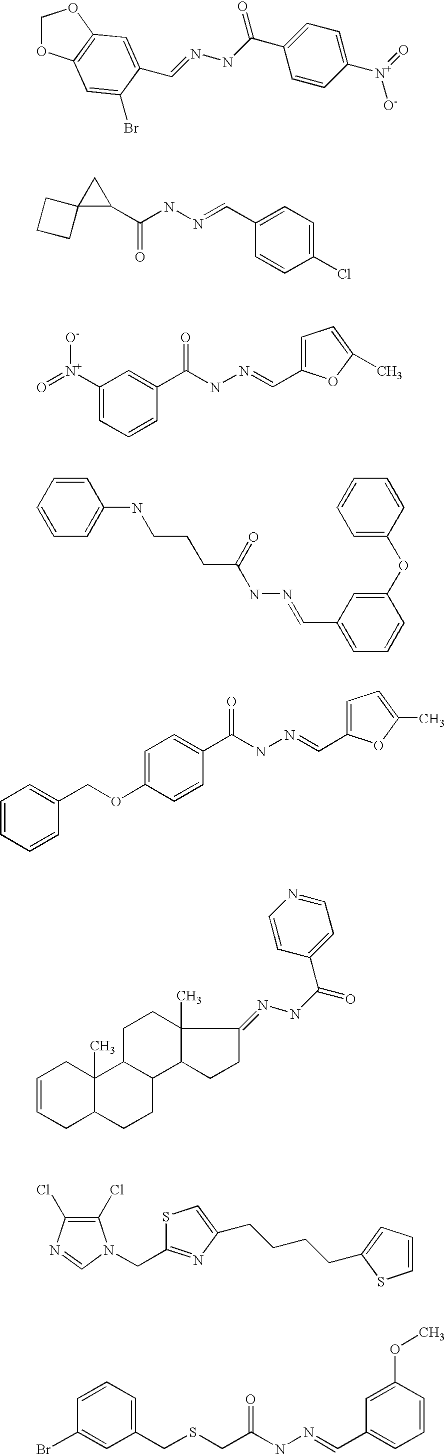 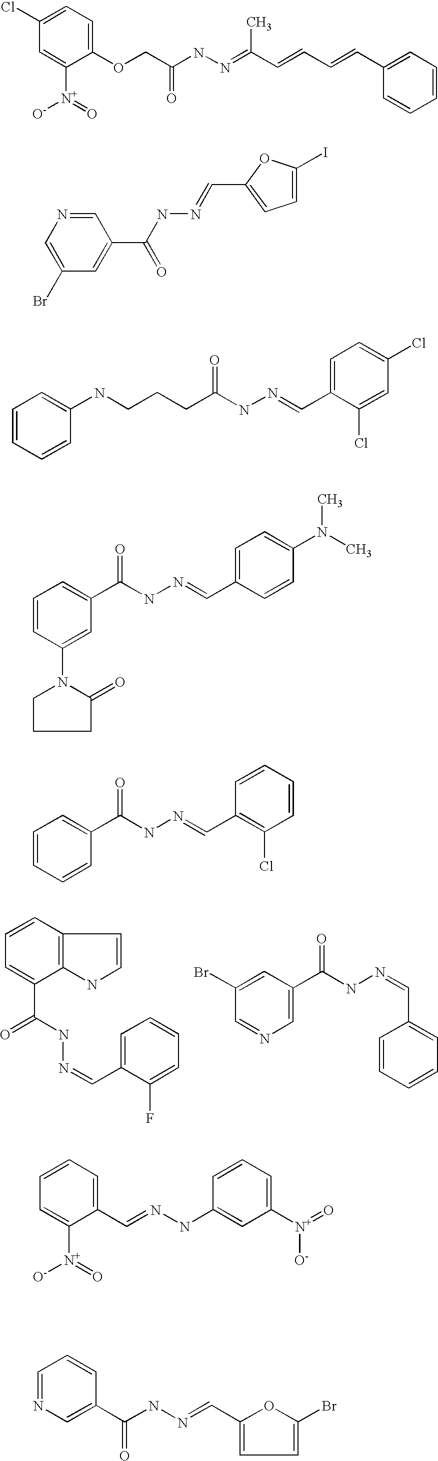 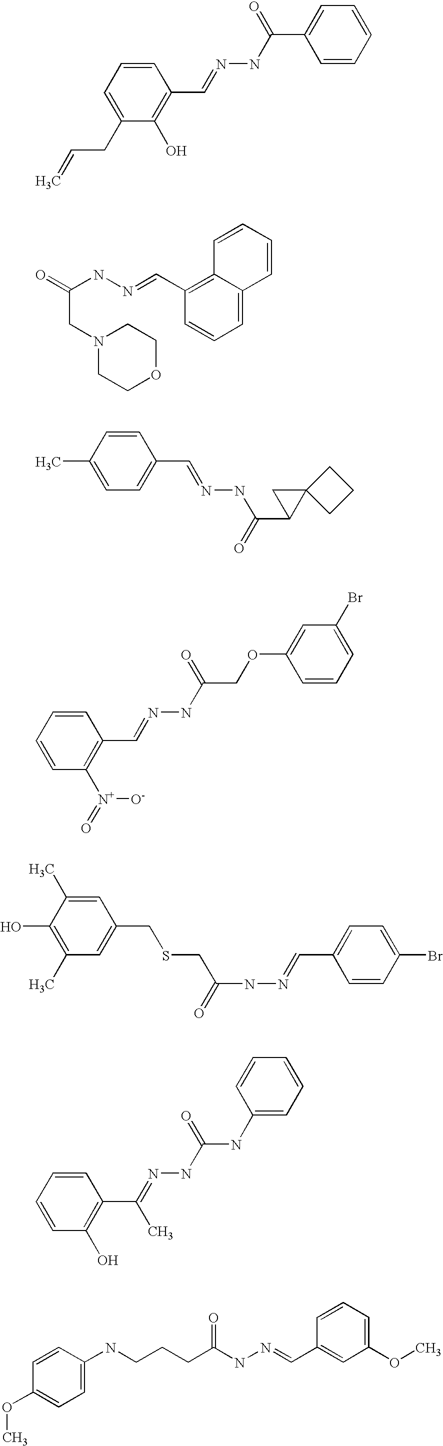 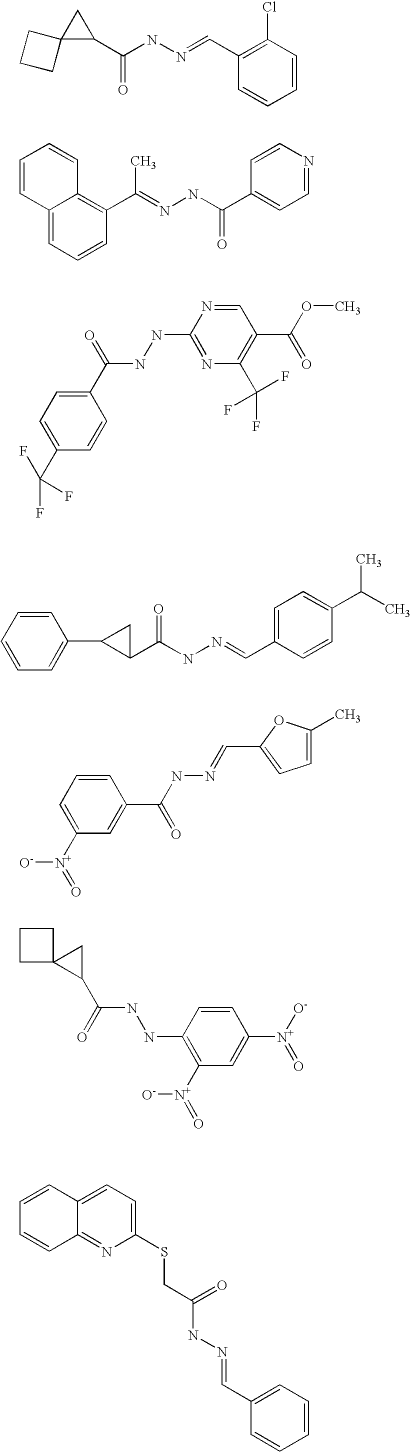 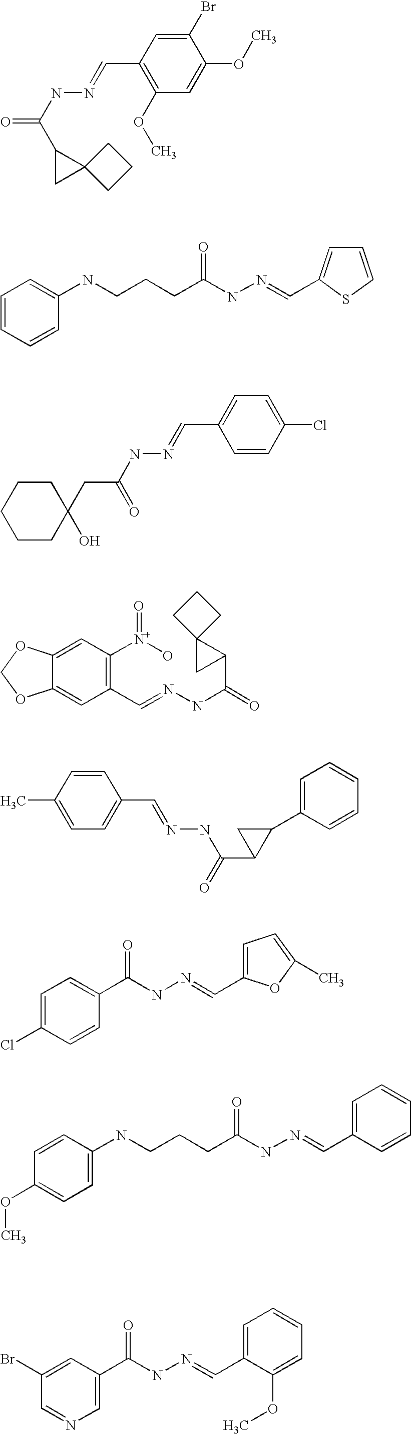 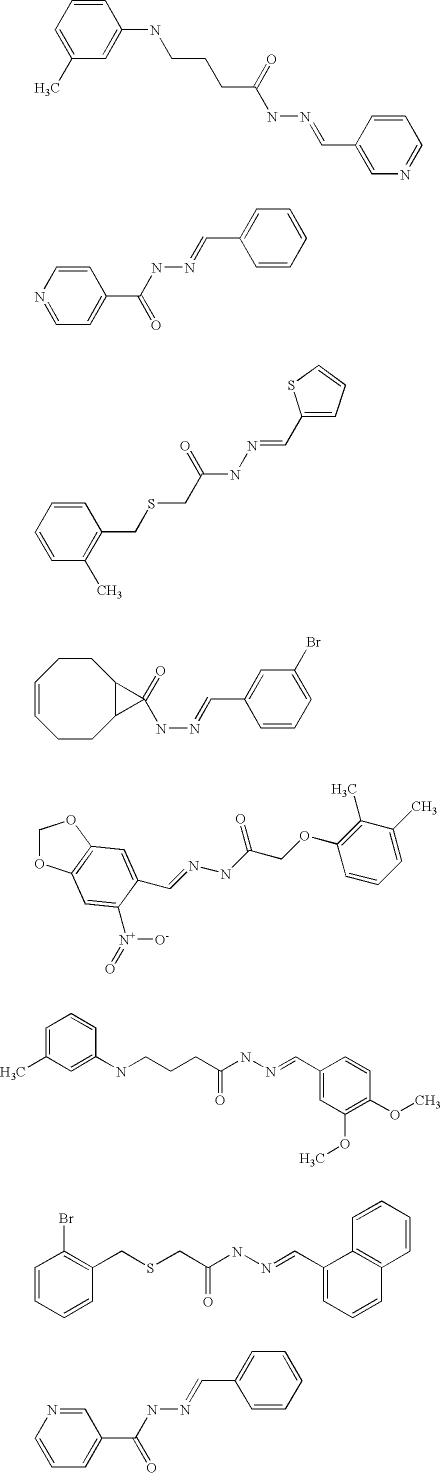 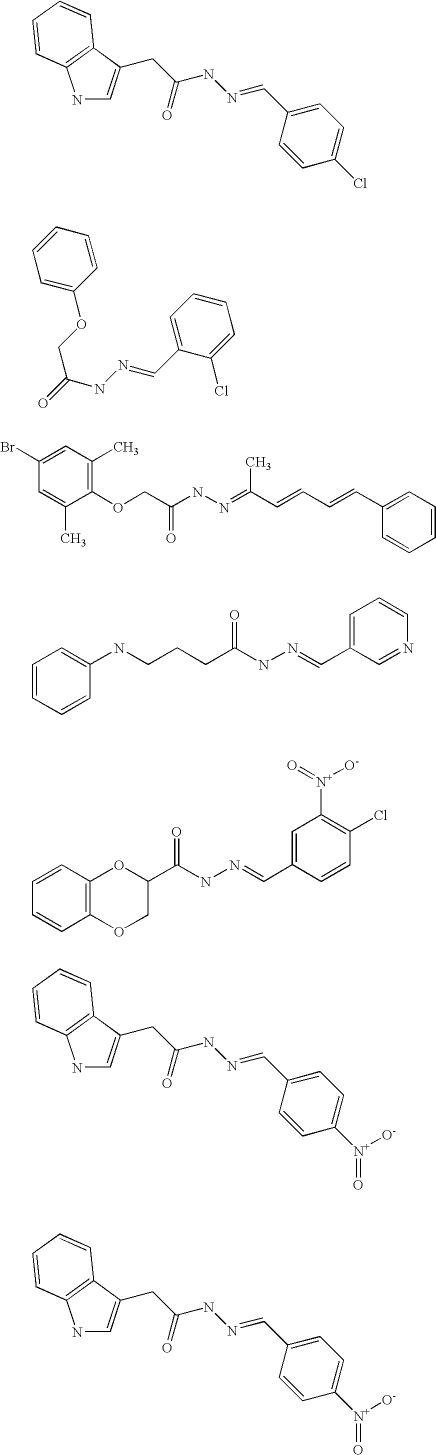 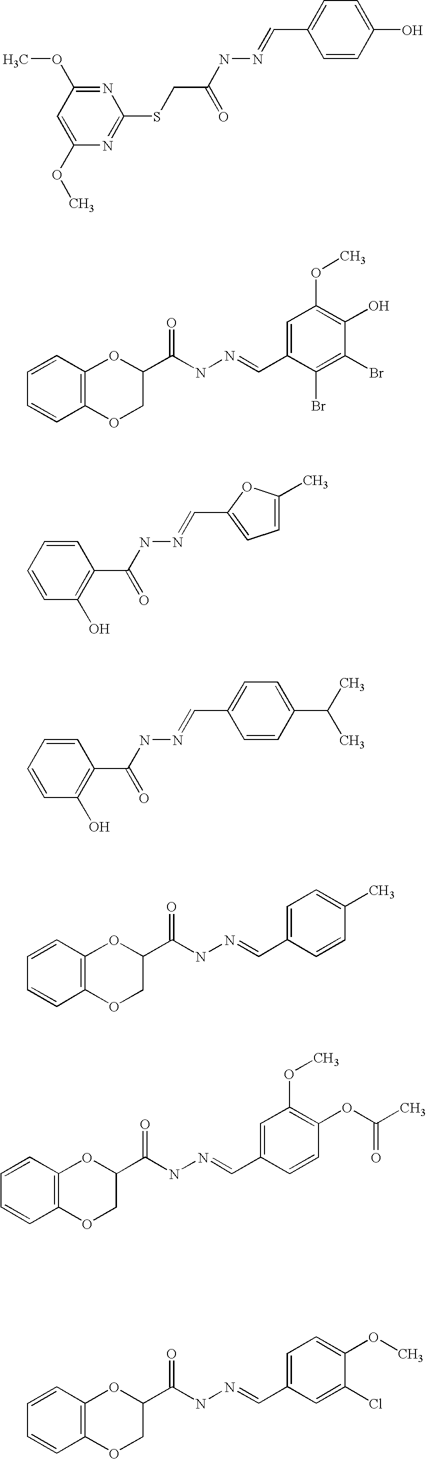 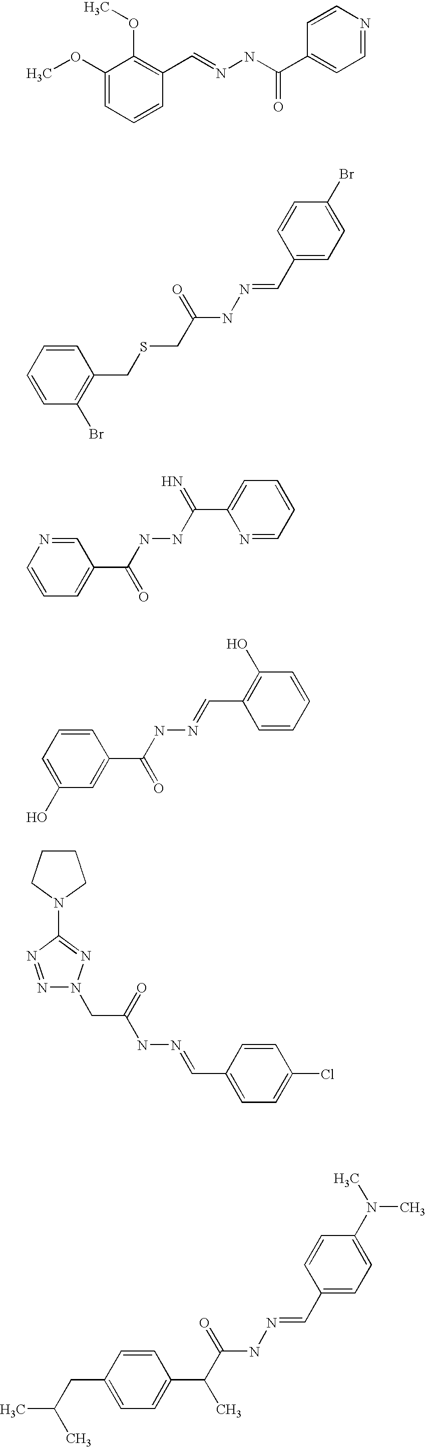
or the pharmaceutically acceptable salts thereof

General Synthetic Procedures:

The above compounds can be prepared using the following synthetic schemes and with methods known in the art.

Compounds of formula (I) or (II) may be prepared using synthetic Schemes 4,5 or 6

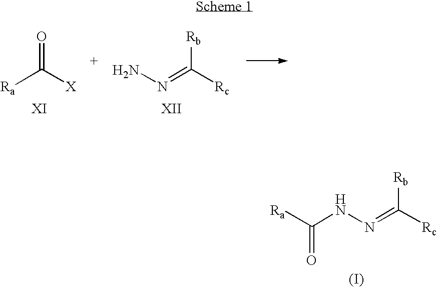

As illustrated in Scheme 1, reacting an acylating agent, such as acid halides, acid anhydrides or esters, of the formula XI with a hydrazine derivative of formula XII, in a suitable solvent, provides a compound of formula (I). The starting materials of formula XI and XII are either commercially available or may be prepared by methods known in the literature or to one skilled in the art.

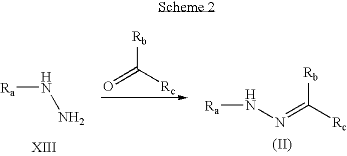

As illustrated in Scheme 2, reacting the alkyl or aryl derivative of a hydrazine of formula XIII with a carbonyl compound, in a suitable solvent, provides a compound of formula (I). The starting hydrazine derivatives as well as the carbonyl compounds are either commercially available or may be prepared by methods known in the literature or to one skilled in the art.

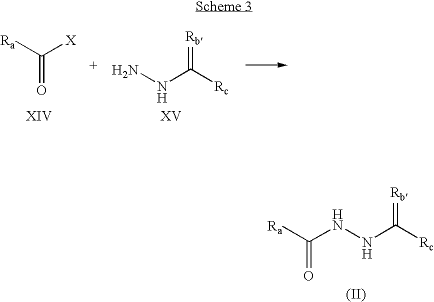

As illustrated in Scheme 3, reacting an acylating agent, such as acid halides, acid anhydrides or esters, of the formula XIV with a hydrazine derivative of formula XV, in a suitable solvent, provides a compound of formula (II). The starting materials of formula XIV and XV are either commercially available or may be prepared by methods known in the literature or to one skilled in the art.

For any of the above mentioned embodiments, cardiovascular diseases shall include: type 1 and type 2 diabetes, insulin resistance syndrome, hypertension, atherosclerosis, coronary artery disease, angina, ischemia, ischemic stroke, Raynaud's disease or renal disease.

Any of the compounds described above include all isomeric forms of these compounds which are expressly included in the present invention. The term ‘isomer’ is defined herein below.

All terms as used herein in this specification, unless otherwise stated, shall be understood in their ordinary meaning as known in the art.

Alkyl is a C1-24 carbon chain, optionally saturated or unsaturated to varying degrees, optionally partially or fully halogenated and branched or unbranched.

Carbocycles include hydrocarbon rings containing from three to twenty four carbon atoms. These carbocycles may be either aromatic either aromatic or non-aromatic ring systems. The non-aromatic ring systems may be mono- or polyunsaturated. Polycyclic carbocycles possessing two, three or four rings are also included in this definition such as naphthalene, indene, azulene, fluorene, phenanthrene, anthracene, acenaphthalene, biphenylene, as are those forming a cyclopentanohydrophenanthrene ring system, or those possessing bridged ring systems. Each of the above may contain a spiro carbon including another carbocyclic or heterocyclic ring system. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Other representative carbocycles include:

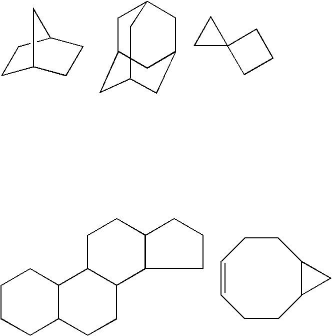

All carbocycles are optionally partially unsaturated, optionally partially or fully halogenated and optionally substituted. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic, or 10-24 membered tricyclic or tetracyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure. Each may be bnzofused to a phenyl ring (which is optionally fully or partially saturated). Unless otherwise stated, heterocycles include but are not limited to, for example pyrrolidinyl, pyrrolinyl, morpholinyl, thiomorpholinyl, thiomorpholinyl sulfoxide, thiomorpholinyl sulfone, dioxalanyl, piperidinyl, piperazinyl, tetrahydrofuranyl, tetrahydropyranyl, tetrahydrofuranyl, 1,3-dioxolanone, 1,3-dioxanone, 1,4-dioxanyl, piperidinonyl, tetrahydropyrimidonyl, pentamethylene sulfide, pentamethylene sulfoxide, pentamethylene sulfone, tetramethylene sulfide, tetramethylene sulfoxide and tetramethylene sulfone.

The term “heteroaryl” shall be understood to mean an aromatic 5-8 membered monocyclic or 8-11 membered bicyclic ring containing 1-4 heteroatoms such as N, O and S. Unless otherwise stated, such heteroaryls include aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazolo[3,4-b]pyridinyl, tubercidinyl, oxazo[4,5-b]pyridinyl and imidazo[4,5-b]pyridinyl.

The term “heteroatom” as used herein shall be understood to mean atoms other than carbon such as O, N, S and P.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative. For example, quinolinyl may include decahydroquinolinyl and tetrahydroquinolinyl, naphthyl may include it's hydrogenated derivatives such as tetrahydranaphthyl. Other partially or fully hydrogenated derivatives of the aryl and heteroaryl compounds described herein will be apparent to one of ordinary skill in the art.

As used herein, “nitrogen” and “sulfur” include any oxidized form of nitrogen and sulfur and the quaternized form of any basic nitrogen. For example, for an —S—C₁₋₆ alkyl radical, unless otherwise specified, this shall be understood to include —S(O)—C₁₋₆ alkyl and —S(O)₂—C₁₋₆ alkyl.

As used herein, “amine” is —NH₂ and may be independently mono- or di-substituted by alkyl, aryl, arylalkyl or hydroxyl.

The term “halogen” as used in the present specification shall be understood to mean bromine, chlorine, fluorine or iodine, preferably fluorine. The definitions “partially or fully halogenated“; partially or fully fluorinated; “substituted by one or more halogen atoms”, includes for example, mono, di or tri halo derivatives on one or more carbon atoms. For alkyl, a nonlimiting example would be —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

Pharmaceutically Acceptable Derivative:

A “pharmaceutically acceptable derivative” refers to any pharmaceutically acceptable salt or ester of a compound of this invention, or any other compound which, upon administration to a patient, is capable of providing (directly or indirectly) a compound used in this invention, a pharmacologically active metabolite or pharmacologically active residue thereof. Pharmaceutically acceptable derivatives include prodrugs or prodrug derivatives, solvates, isomers as defined herein and combinations thereof.

The terms “prodrug” or “prodrug derivative” mean a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987, each of which is incorporated herein by reference in their entireties.

The term “pharmaceutically acceptable prodrug” as used herein means a prodrug of a compound of the invention which is, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible.

The term “salt” means an ionic form of the parent compound or the product of the reaction between the parent compound with a suitable acid or base to make the acid salt or base salt of the parent compound. Salts of the compounds of the present invention can be synthesized from the parent compounds which contain a basic or acidic moiety by conventional chemical methods. Generally, the salts are prepared by reacting the free base or acid parent compound with stoichiometric amounts or with an excess of the desired salt-forming inorganic or organic acid or base in a suitable solvent or various combinations of solvents.

The term “pharmaceutically acceptable salt” means a salt of a compound of the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. As the compounds of the present invention are useful in both free base and salt form, in practice, the use of the salt form amounts to use of the base form. Lists of suitable salts are found in, e.g., S. M. Birge et al., J. Pharm. Sci., 1977, 66, pp. 1-19, which is hereby incorporated by reference in its entirety.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, hydroiodic acid, sulfuric acid, sulfamic acid, nitric acid, phosphoric acid, and the like, and organic acids such as acetic acid, trichloroacetic acid, trifluoroacetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, heptanoic acid, hexanoic acid, formic acid, fumaric acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N-dibenzylethylenediamine, polyamine resins, and the like. Particularly preferred organic nontoxic bases are isopropylamine, diethylamine, ethanolamine, trimethylamine, dicyclohexylamine, choline, and caffeine.

The term “solvate” means a physical association of a compound with one or more solvent molecules or a complex of variable stoichiometry formed by a solute (for example, a compound of Formula (I)) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompasses both solution-phase and isolatable solvates. Representative solvates include hydrates, ethanolates, methanolates, and the like.

The term “hydrate” means a solvate wherein the solvent molecule(s) is/are H₂O.

The compounds of the present invention as discussed below include the free base or acid thereof, their salts, solvates, and prodrugs and may include oxidized sulfur atoms or quaternized nitrogen atoms in their structure, although not explicitly stated or shown, particularly the pharmaceutically acceptable forms thereof. Such forms, particularly the pharmaceutically acceptable forms, are intended to be embraced by the appended claims.

Isomer Terms and Conventions

The term “isomer” includes stereoisomers and geometric isomers.

The term “stereoisomer” means a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the invention which may give rise to optical isomerism, the invention contemplates stereoisomers and mixtures thereof. The compounds of the invention and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure optical isomers, i.e., as individual enantiomers or diastereomers, or as stereoisomer-enriched mixtures. Individual stereoisomers of compounds are prepared by synthesis from optically active starting materials containing the desired chiral centers or by preparation of mixtures of enantiomeric products followed by separation, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

The term “enantiomers” means a pair of optical isomers that are non-superimposable mirror images of each other.

The terms “diastereoisomers” or “diastereomers” mean stereoisomers which are not mirror images of each other.

The terms “racemic mixture” or “racemate” mean a mixture containing equal parts of individual enantiomers.

The term “non-racemic mixture” means a mixture containing unequal parts of individual enantiomers.

The term “geometrical isomer” means a stable isomer which results from restricted freedom of rotation about double bonds (e.g., cis-2-butene and trans-2-butene) or in a cyclic structure (e.g., cis-1,3-dichlorocyclobutane and trans-1,3-dichlorocyclobutane). Because carbon-carbon double (olefinic) bonds, C═N double bonds, cyclic structures, and the like may be present in the compounds of the invention, the invention contemplates each of the various stable geometric isomers and mixtures thereof resulting from the arrangement of substituents around these double bonds and in these cyclic structures. The substituents and the isomers are designated using the cis/trans convention or using the E or Z system, wherein the term “E” means higher order substituents on opposite sides of the double bond, and the term “Z” means higher order substituents on the same side of the double bond. A thorough discussion of E and Z isomerism is provided in J. March, Advanced Organic Chemistry: Reactions Mechanisms and Structure, 4th ed., John Wiley & Sons, 1992, which is hereby incorporated by reference in its entirety. Several of the following examples represent single E isomers, single Z isomers, and mixtures of E/Z isomers. Determination of the E and Z isomers can be done by analytical methods such as x-ray crystallography, ¹H NMR, and ¹³C NMR.

Some of the compounds of the invention can exist in more than one tautomeric form. As mentioned above, the compounds of the invention include all such tautomers.

In general, all tautomeric forms and isomeric forms and mixtures, whether individual geometric isomers or optical isomers or racemic or non-racemic mixtures, of a chemical structure or compound is intended, unless the specific stereochemistry or isomeric form is specifically indicated in the compound name or structure.

Pharmaceutical Administration and Diagnostic and Treatment Terms and Conventions

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

The terms “treating” or “treatment” mean the treatment of a disease-state in a patient, and include:
(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

The compounds described herein are either commercially available or can be made by methods and any necessary intermediates well known in the art.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the scheme below are either commercially available or easily prepared from commercially available materials by those skilled in the art.

### Method of Use

In accordance with the invention, there are provided methods of using the compounds as desrcribed herein and their pharmaceutically acceptable derivatives. The compounds used in the invention prevent the degradation of sEH substrates that have beneficial effects or prevent the formation of metabolites that have adverse effects. The inhibition of sEH is an attractive means for preventing and treating a variety of cardiovascular diseases or conditions e.g., endothelial dysfunction. Thus, the methods of the invention are useful for the treatment of such conditions. These encompass diseases including, but not limited to, type 1 and type 2 diabetes, insulin resistance syndrome, hypertension, atherosclerosis, coronary artery disease, angina, ischemia, ischemic stroke, Raynaud's disease and renal disease.

For therapeutic use, the compounds may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds described herein may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the above-described compounds include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

Fluorescence Polarization Assay to Determine Inhibition of sEH:

Step one: Characterization of the Fluorescent Probe

The wavelengths for maximum excitation and emission of the fluorescent probe should first be measured. An example of such a probe is compound (4) as shown in WO 02/082082, where these values are 529 nm and 565 nm, respectively. These fluorescence wavelength values were measured on an SLM-8100 fluorimeter with the probe dissolved in an assay buffer (20 mM TES, pH 7.0, 200 mM NaCl, 0.05% (w/v) CHAPS, 2 mM DTT).

The affinity of the probe for sEH was then determined in a titration experiment. The fluorescence polarization value of compound 4 in assay buffer was measured on an SLM-8100 fluorimeter using the excitation and emission maximum values described above. Aliquots of sEH were added and fluorescence polarization was measured after each addition until no further change in polarization value was observed. Non-linear least squares regression analysis was used to calculate the dissociation constant of compound 4 from the polarization values obtained for sEH binding to compound 4. FIG. 1 shows the results from this titration experiment

Step two: Screening for inhibitors of probe binding

In order to screen a large number of compounds the assay was performed using a 96-well plate format. An example of such a plate is the Dynex Microfluor 1, low protein binding U -bottom black 96 well plates (#7005). The plate is set up by first creating a complex between recombinant human sEH and a fluorescent probe that binds to the active site of sEH. In this example, the complex between compound 4 and sEH, was pre-formed in assay buffer (20 mM TES, pH 7.0, 200 mM NaCl, 0.05% (w/v) CHAPS, 1 mM TCEP). The concentrations of sEH and compound 4 in this solution were made up such that the final concentration in the assay was 10 nM sEH and 2.5 nM compound 4. Test compounds were then serially diluted into assay buffer, across a 96 well plate. The pre-formed sEH-probe complex was then added to all the wells and incubated for 15 minutes at room temperature. The fluorescence polarization was then measured using a fluorescence polarization plate reader set at the wavelengths appropriate for the fluorescent label on the fluorescent probe (4). In this example, an LJL Analyst was set to read rhodamine fluorescence polarization (Ex 530 nM, Em 580 nM). Dissociation constants (Kd) were calculated as described in WO 02/082082, for the test compounds binding to sEH from the polarization values for the probe binding to sEH in the presence of the test compounds.

An observed decrease in fluorescence polarization of the probe-sEH complex in the presence of the test compound is evidence that this test compound is an inhibitor of soluble epoxide hydrolase that competes with the fluorescent probe for the sEH active site binding.

Preferred compounds have a calculated Kd below 1 micromolar. More preferred compounds have a calculated Kd below 100 nM. Most preferred compounds have a calculated Kd below 10 nM.

What is claimed:

1. A method of treating a disease or condition chosen from type 1 and type 2 diabetes, insulin resistance syndrome, hypertension, atherosclerosis, coronary artery disease, angina, ischemia, ischemic stroke, Raynaud's disease and renal disease, said method comprising administering to a patient a therapeutically effective amount of a compound of the formulas (I) or (II): 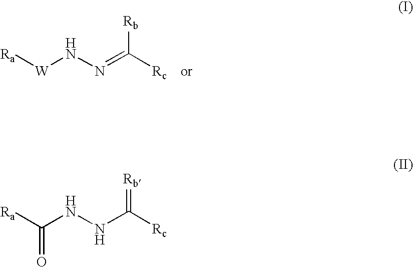
W is a bond or >C═O;
Rₐ and R_{c} are
—Ar₁, —(Ar₂)ₜ—(CH₂)_{q}—X₁—(CH₂)ₙ—X₂—(CH₂)ₘ—Y wherein Y is —CH₃ or Ar₁,
Ar₁ and Ar₂ are each independently a heterocylic or carbocyclic ring system,
each X₁ and X₂ are independently a bond, >C═O, O, NH, NR or S(O)ₚ;
m, n and q are 0-5;
t is 0 or 1;
p is 0-2;
each alkyl chain formed by —(CH₂)_{q}—, —(CH₂)ₙ—, —(CH₂)ₘ— can be saturated or partially or fully unsaturated;
R_{b}′ is ═O, ═NH, ═CH₂,
R_{b} is hydrogen or C₁₋₅ alkyl,
or R_{b} and R_{c} fuse to form a 3-17 carbon carbocyclic or a 4-17 carbon heterocyclic ring system, each ring system being mono-, bicyclic-,tricyclic or tetracyclic
each of the aforementioned rings in this embodiment is optionally substituted by one or more halogen, nitro, amine, C₁₋₅ alkyl, C₁₋₅ alkoxy or hydroxyl;
or the pharmaceutically acceptable salts thereof

2. The method according to claim 1 wherein:
Ar₁ and Ar₂ are chosen from:
phenyl, thienyl, morpholine, quinoline, piperidine, bicycle[2.2.1]heptane, adamantly, pyrrolidine, pyrrolidinone, pyridine, naphthylene, benzthiophene, furan, imidazole, benzodioxolanyl, benzodioxanyl, indole, indane, piperazine, thiazole, pyrimidine, cyclopropyl, cyclobutyl, cyclopentyl, cyclohexyl, tetrazole 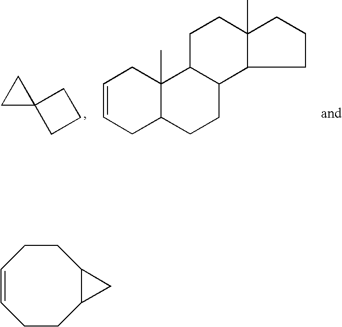
and each Ar₁ and Ar₂ is optionally substituted by one or more halogen, nitro, amine, C₁₋₅ alkyl, C₁₋₅ alkoxy or hydroxyl.

3. A method of treating a disease or condition chosen from type 1 and type 2 diabetes, insulin resistance syndrome, hypertension, atherosclerosis, coronary artery disease, angina, ischemia, ischemic stroke, Raynaud's disease and renal disease, said method comprising administering to a patient a therapeutically effective amount of one or more compounds chosen from: 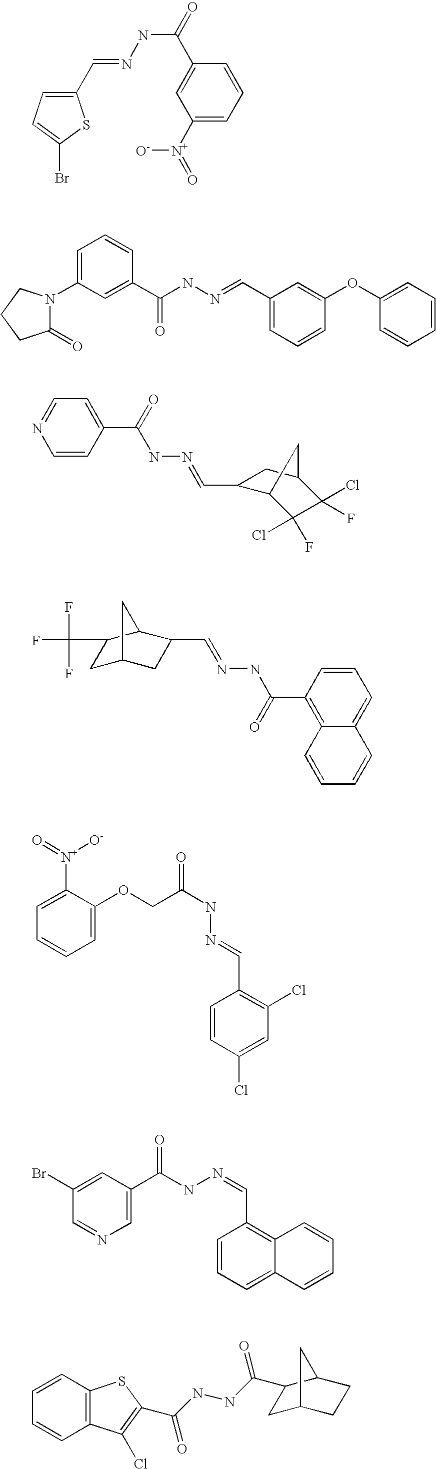 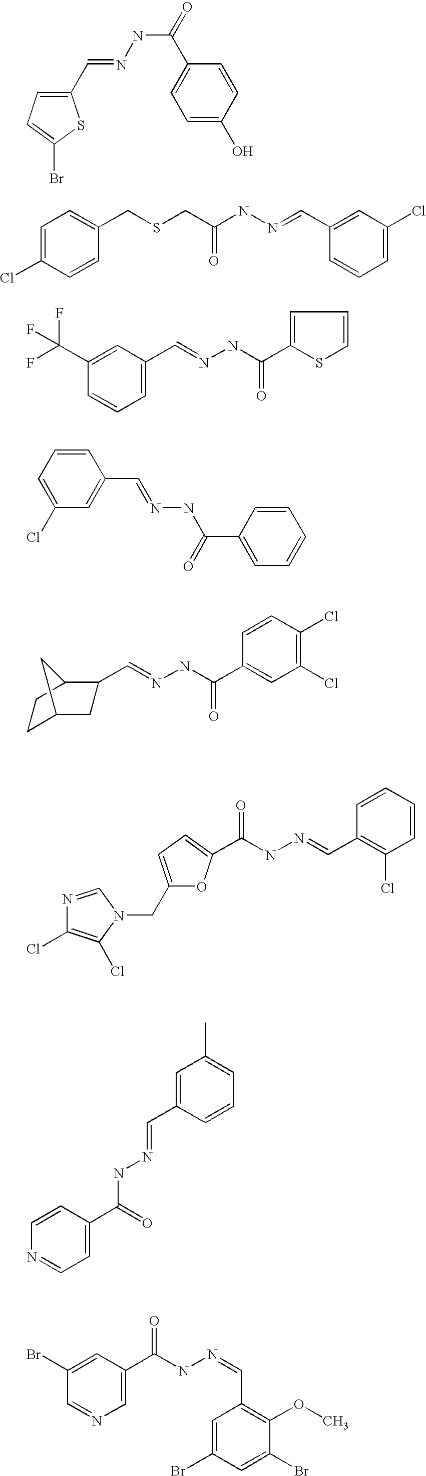 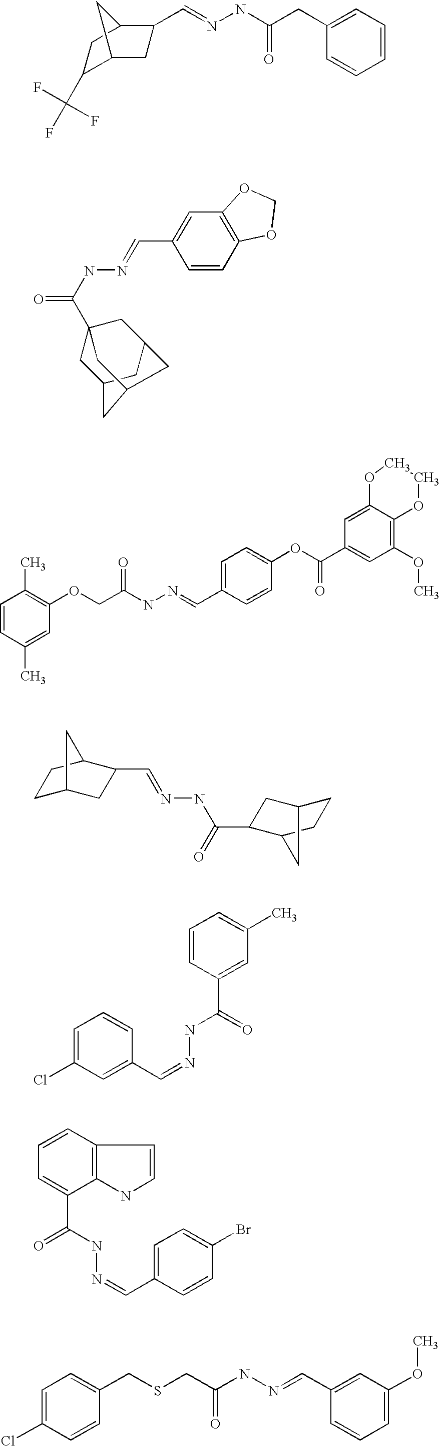 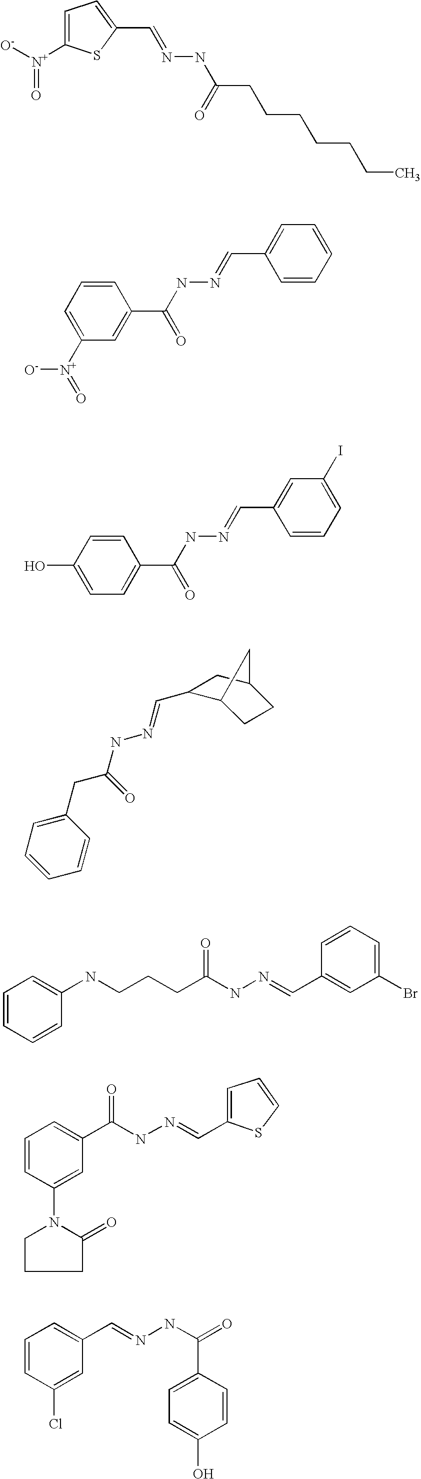 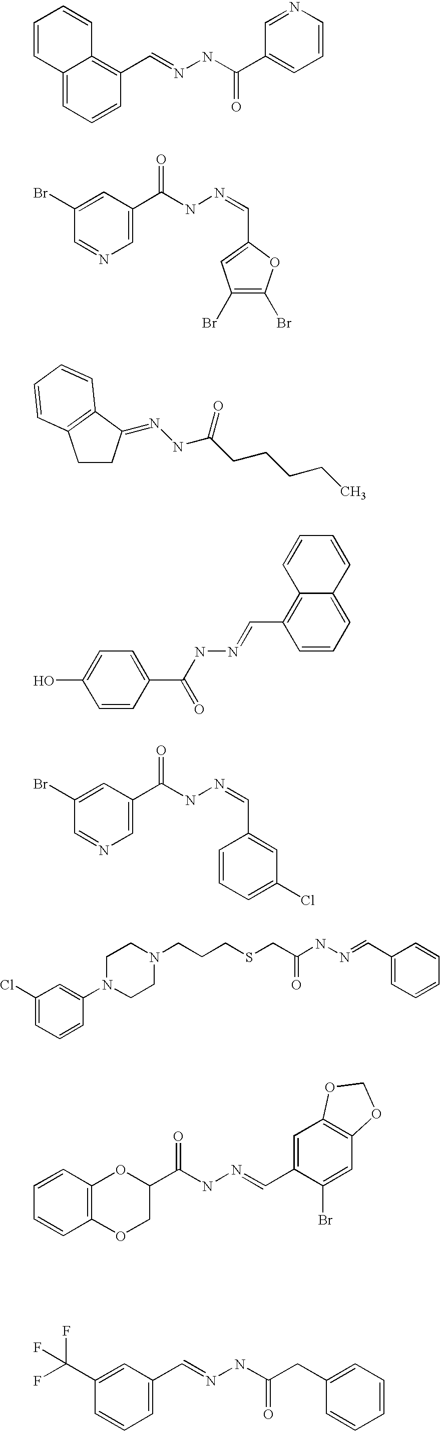 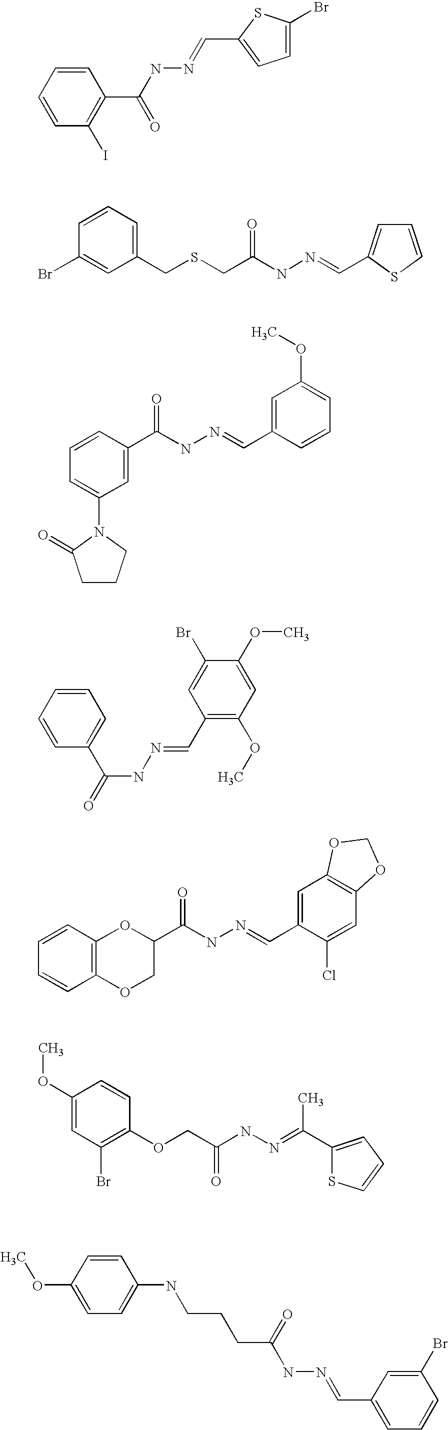 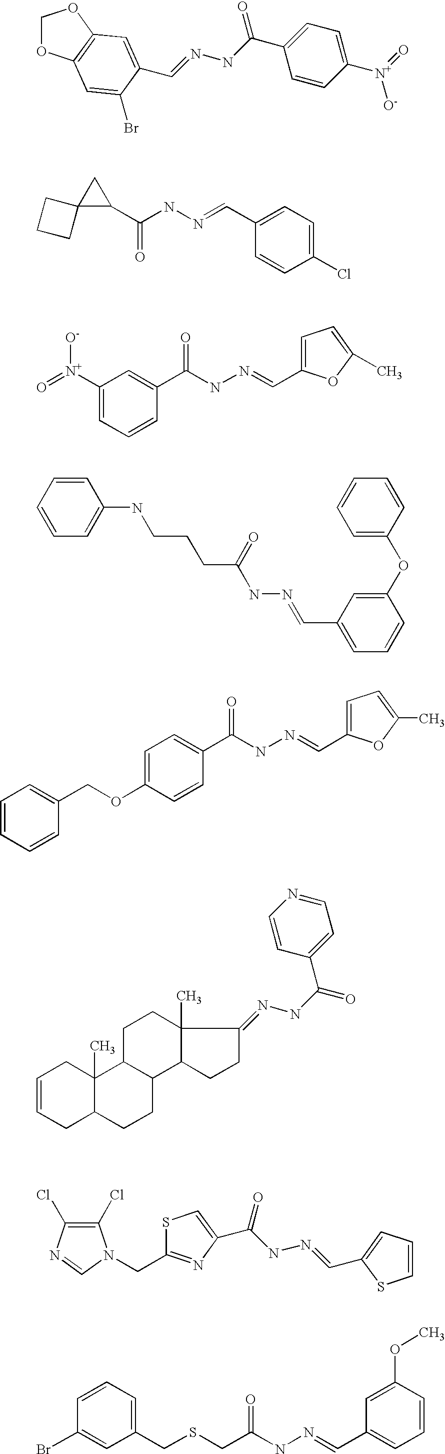 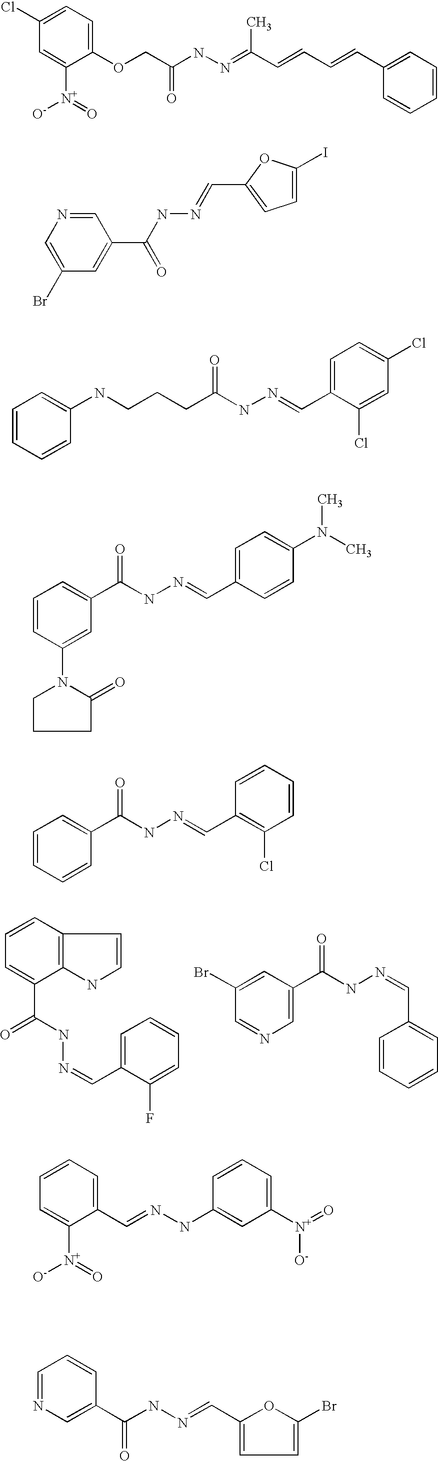 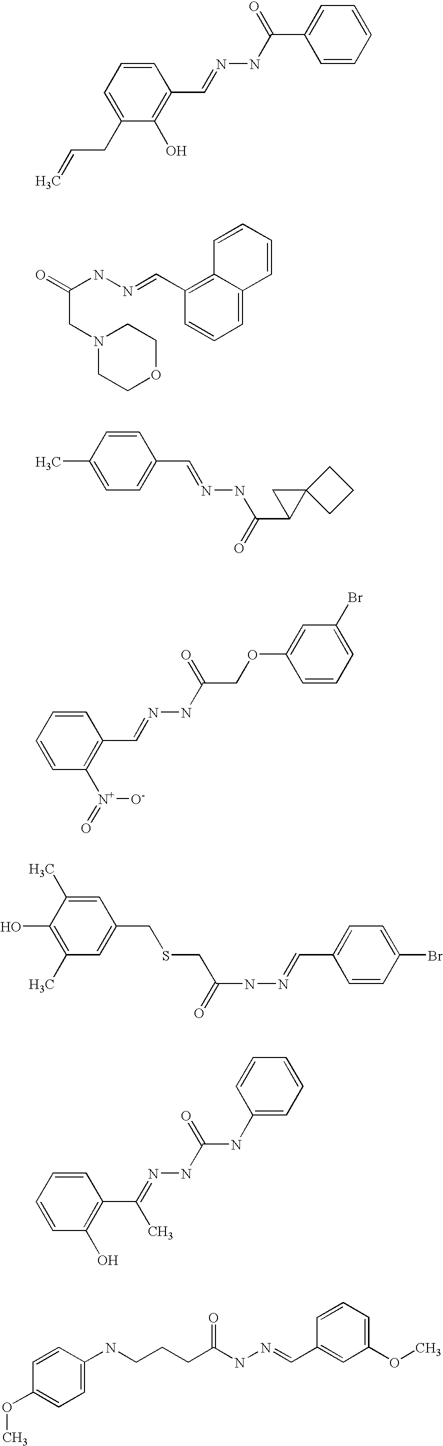 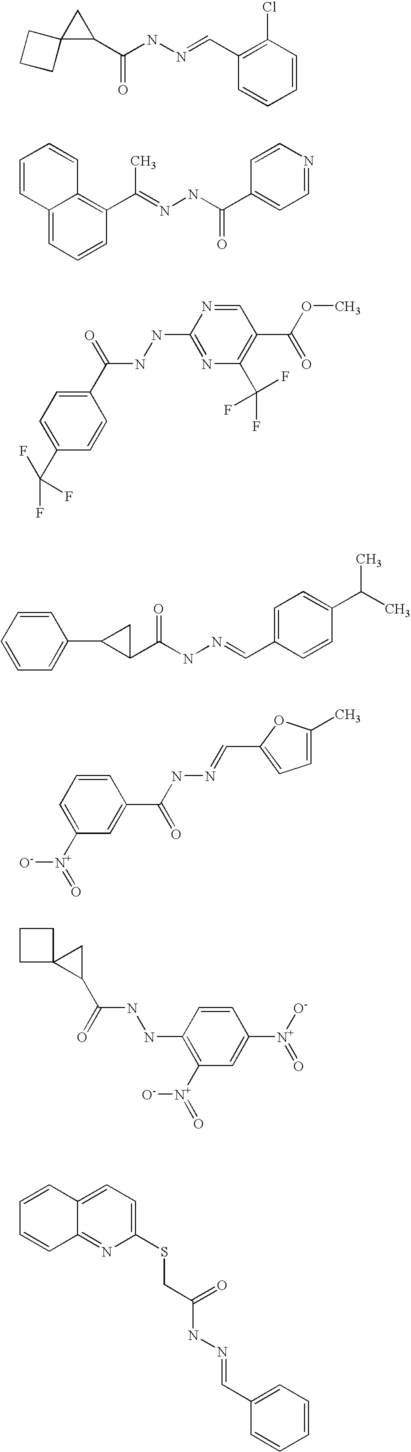 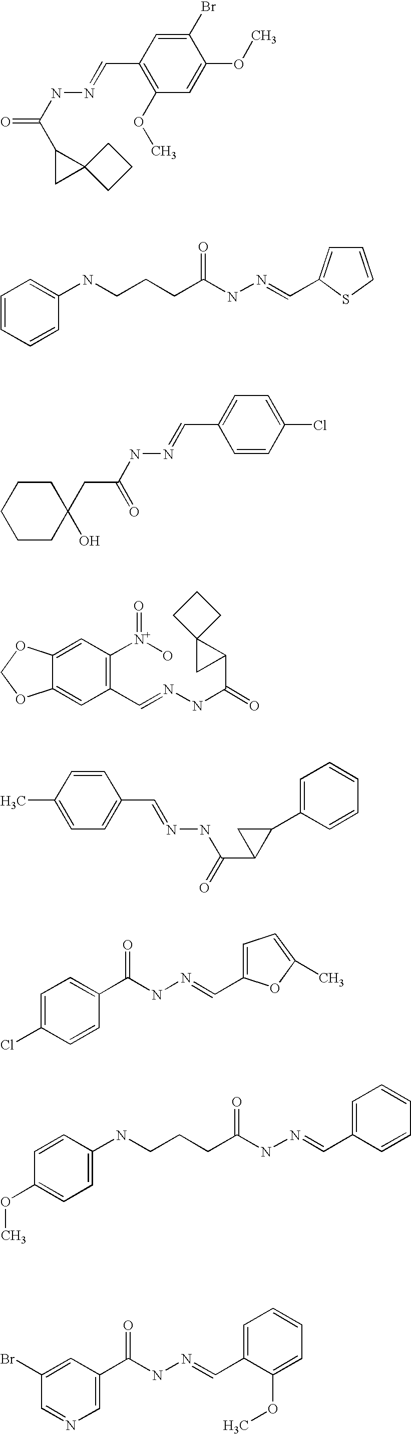 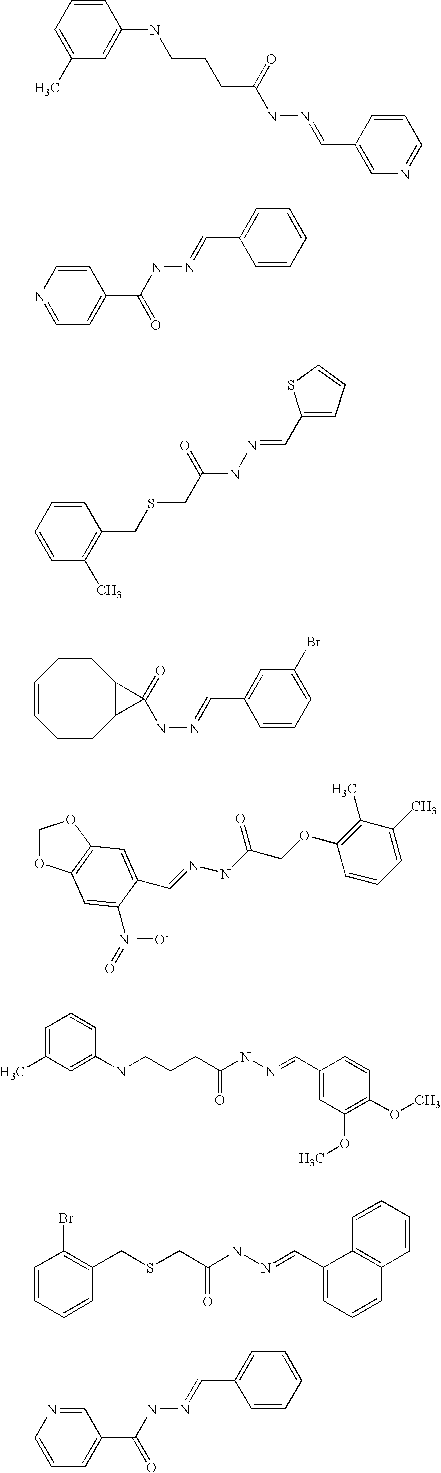 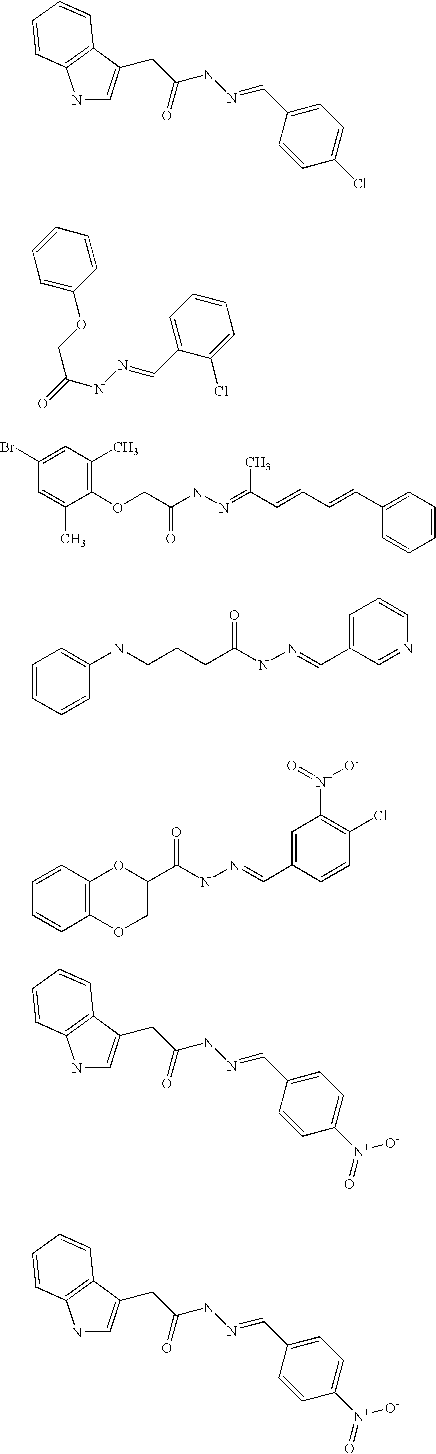 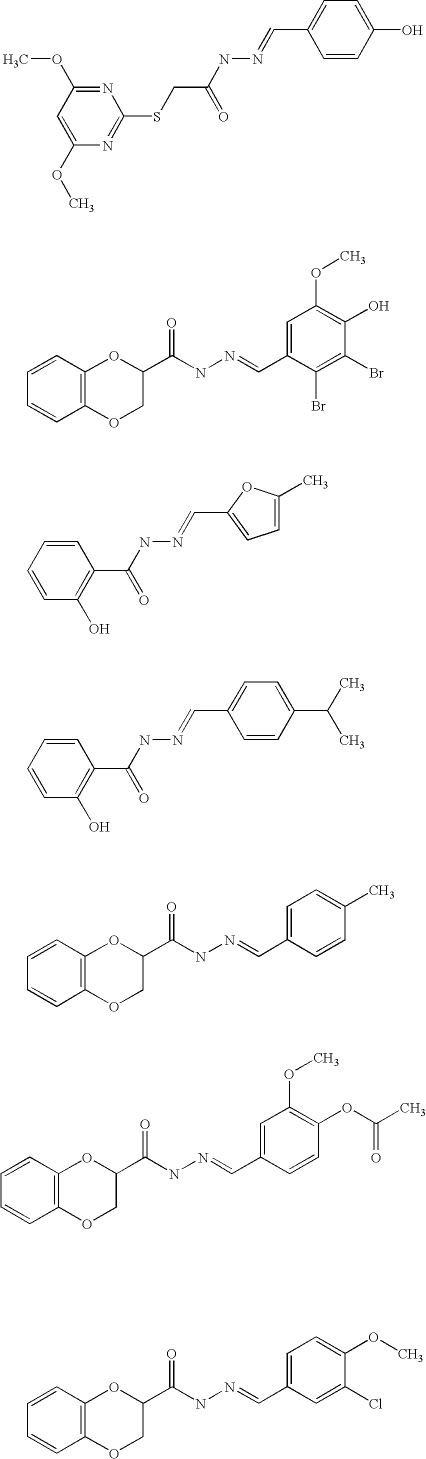 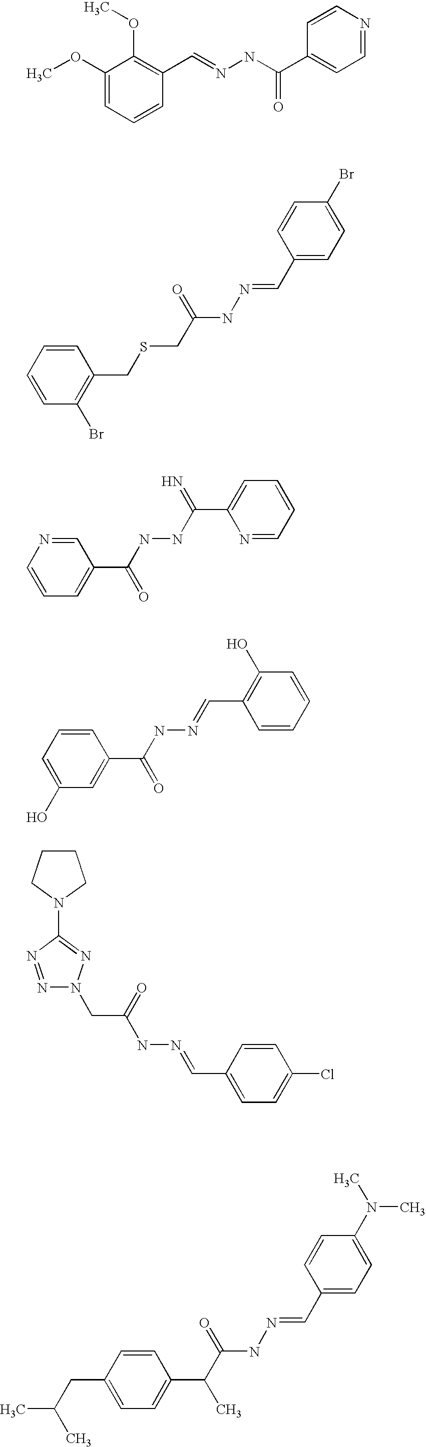
or the pharmaceutically acceptable salts thereof
